(19) United States
(12) Patent Application Publication
(10) Pub. No.: US 2017/0056311 A1
(43) Pub. Date: Mar. 2, 2017

(54) HIGH-PERFORMANCE HAIR TREATMENT AGENT COMPIRISING AN OLIGOPEPTIDE AND A MAGNESIUM SALT

(71) Applicant: Henkel AG & Co. KGaA, Duesseldorf (DE)

(72) Inventors: Erik Schulze zur Wiesche, Hamburg (DE); Rene Krohn, Norderstedt (DE)

(73) Assignee: Henkel AG & Co. KGaA, Duesseldorf (DE)

(21) Appl. No.: 15/243,514

(22) Filed: Aug. 22, 2016

(30) Foreign Application Priority Data
Sep. 1, 2015 (DE) 10 2015 216 690.2

(51) Int. Cl. A61K 8/64; A61Q 5/12; A61Q 5/02; A61K 8/365; A61Q 5/00

(52) U.S. Cl. CPC A61K 8/64; A61K 8/365; A61Q 5/004; A61Q 5/02; A61Q 5/12; A61Q 5/00

(57) ABSTRACT

Hair treatment agents having a pH of between 3.5 and 5.5 can be obtained by mixing
a) to 20 wt. %—based on the total weight of the hair treatment agent—of at least one oligopeptide that comprises at least one Glu-Glu-Glu amino acid sequence,

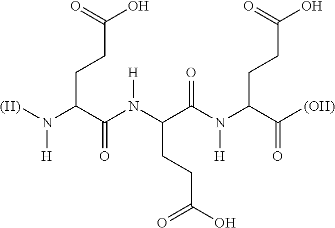

where the amino group may be in free or protonated form and the carboxyl groups may be in free or deprotonated form, and
b) 0.01 to 5.0 wt. %—based on the total weight of the hair treatment agent—of magnesium citrate in a hydrous carrier.
   Hair treatment agents prepared in this way have an increased positive influence on the hair and the hair follicle, and an improved color protection performance when used on artificially produced hair colors.

### FIELD OF THE INVENTION

The present invention generally relates to hair treatment agents, and more particularly relates to shampoos and conditioners, comprising an active ingredient combination for gentle and effective haircare.

### BACKGROUND OF THE INVENTION

Care products having an effect that lasts for as long as possible are becoming increasingly important, not least because of significant stresses to which hair is subjected, for example by coloring or permanent waving as well as by cleansing the hair using shampoos, and by environmental pollution. Care agents of this kind influence the natural structure and the properties of the hair. Thus, following such care treatments, for example the wet and dry combability of the hair, the hold and the body of the hair can be optimized, or the hair can be protected from increased split ends.

It has therefore long been common for hair to undergo a specific subsequent treatment. In this case, the hair is treated, usually in the form of conditioning, with specific active ingredients, for example quaternary ammonium salts or specific polymers. Depending on the formulation, the combability, the hold and the body of the hair are improved and the number of split ends is reduced by this treatment.

Multifunctional cosmetic products are likewise known in the prior art. These include in particular what are known as “2 in 1” shampoos, which not only cleanse the hair but also condition it. Products of this kind are highly valued by consumers since their product performance means that at least one method step, for example conditioning using a conventional hair conditioner, is not necessary.

The use of specific oligopeptides for caring for keratin fibers is known for example from WO 2010/026010 A1. In particular oligopeptides of the sequence Tyr-Glu-Glu-Glu-Ile-Arg-Val-Leu are disclosed here as being particularly suitable for haircare.

Products for changing the natural hair color likewise play a prominent role in hair cosmetics. A distinction is made between permanent, semi-permanent or temporary coloring systems that are based on chemical and/or natural dyes. However, the hair colors artificially produced by permanent, semi-permanent or temporary coloring systems have the disadvantage that they can change in an undesired manner, for example during or after cleansing of the hair.

An “undesired change” is understood as the lightening or bleeding and the loss of the color brilliancy of the hue of the hair achieved by the respective coloring processes. Environmental influences and/or the action of sunlight can further increase these changes.

The use of divalent metal salts in hair dyes for improving the durability and thus the fastness of the coloring process is known from EP 2438900 A1.

There is still the need to provide active ingredients or active ingredient combinations for hair treatment agents that have good caring properties which in addition increase the adherence of dyes to the hair fibers and thus maintain the fastness of the artificially produced hair color, and to develop hair treatment agents in this respect.

Furthermore, other desirable features and characteristics of the present invention will become apparent from the subsequent detailed description of the invention and the appended claims, taken in conjunction with this background of the invention.

### BRIEF SUMMARY OF THE INVENTION

A hair treatment agent having a pH of between 3.5 and 5.5 can be obtained by mixing to 20 wt. %—based on the total weight of the hair treatment agent—of at least one oligopeptide that comprises at least one Glu-Glu-Glu amino acid sequence,

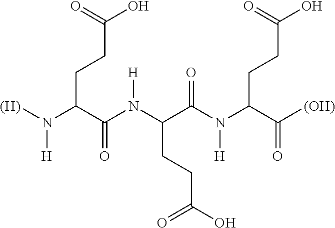

where the amino group may be in free or protonated form and the carboxyl groups may be in free or deprotonated form; and 0.01 to 5.0 wt. %—based on the total weight of the hair treatment agent—of magnesium citrate in a hydrous carrier.

A method for reducing and/or preventing the bleeding and/or fading of artificially obtained hair colors, and/or for improving the color intensity and/or the color fidelity, and for improving at least one of the properties of tensile strength of keratin fibers, in particular human hair; stabilizing the moisture balance of keratin fibers, in particular human hair; combability of keratin fibers, in particular human hair; increasing the contact angle between water droplets and the surface of keratin fibers, in particular human hair; lessening the reduction in elasticity of keratin fibers, in particular human hair, when damaged by atmospheric influences, includes applying to the keratin fibers a hair treatment agent that has a pH of between 3.5 and 5.5 and can be obtained by mixing to 20 wt. %—based on the total weight of the hair treatment agent—of at least one oligopeptide that comprises at least one Glu-Glu-Glu amino acid sequence,

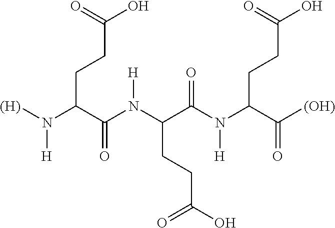

wherein the amino group may be in free or protonated form and the carboxyl groups may be in free or deprotonated form; and 0.01 to 5.0 wt. %—based on the total weight of the hair treatment agent—of magnesium citrate in a hydrous carrier. The agent is left there for between 5 seconds and 10 minutes, and then rinsed out. Alternatively the agent is applied to the keratin fibers and remains there.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that a combination of specific oligopeptides with magnesium citrate has a particularly positive effect on colored hair that is treated therewith and on the hair follicle.

The present invention firstly relates to hair treatment agents having a pH of between 3.5 and 5.5 that can be obtained by mixing
a) to 20 wt. %—based on the total weight of the hair treatment agent—of at least one oligopeptide that comprises at least one Glu-Glu-Glu amino acid sequence,

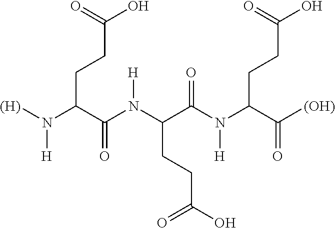

where the amino group may be in free or protonated form and the carboxyl groups may be in free or deprotonated form, and
b) 0.01 to 5.0 wt. %—based on the total weight of the hair treatment agent—of magnesium citrate in a hydrous carrier.

In this as in all the formulae below, the bracketed hydrogen atom of the amino group and the bracketed hydroxyl group of the acid function mean that the groups in question may be present as such (this then making it an oligopeptide having the relevant number of amino acids as shown—three amino acids in the above formula), or else that the amino acid sequence exists in an oligopeptide which comprises further amino acids—depending on where the further amino acid(s) is/are bonded, the bracketed constituents of the above formula are replaced by the further amino acid residue(s).

Hair treatment agents within the meaning of the present invention are for example hair shampoos, hair-conditioning agents, conditioning shampoos, hairsprays, hair conditioners, hair masks, hair packs, hair tonics, permanent wave fixing agents, hair coloring shampoos, hair dyes, hair setting agents, hair care agents, hair styling preparations, blow-dry lotions, mousses, hair gels, hair waxes or combinations thereof. In view of the fact that in particular men are often reluctant to use a large number of different agents and/or a large number of application steps, those agents that men already use are preferred. Preferred agents are therefore shampoos, conditioning agents or hair tonics.

The hair treatment agents include—based on their weight—from 0.00001 to 20 wt. % of at least one oligopeptide that comprises at least one Glu-Glu-Glu amino acid sequence, i.e. at least three consecutive glutamic acids.

In the context of the present invention, oligopeptides are condensation products of amino acids which are linked by peptide bonds in the manner of an acid amide and which comprise at least 3 and at most 25 amino acids.

In preferred hair treatment agents, the oligopeptide comprises 5 to 15 amino acids, preferably 6 to 13 amino acids, particularly preferably 7 to 12 amino acids and in particular 8, 9 or 10 amino acids.

The molecular weight of the oligopeptide included in the hair treatment agents can vary depending on whether further amino acids are bound to the Glu-Glu-Glu sequence and depending on the type of these amino acids. Preferred hair treatment agents are characterized in that the oligopeptide has a molecular weight of 650 to 3000 Da, preferably 750 to 2500 Da, particularly preferably 850 to 2000 Da and in particular 1000 to 1600 Da.

In summary, preferred hair treatment agents are characterized in that the oligopeptide used comprises 5 to 15 amino acids, preferably 6 to 13 amino acids, particularly preferably 7 to 12 amino acids and in particular 8, 9 or 10 amino acids, and has a molecular weight of 650 to 3000 Da, preferably 750 to 2500 Da, particularly preferably 850 to 2000 Da and in particular 1000 to 1600 Da.

As can be seen from the preferred number of amino acids in the oligopeptides and the preferred molecular weight range, oligopeptides that do not consist of the three glutamic acids alone, but comprise further amino acids that are bound to this sequence, are preferably used. These further amino acids are preferably selected from certain amino acids, while certain other representatives are less preferred.

In contrast, it is preferred if the oligopeptides used in the hair treatment agents include tyrosine.

It is preferred if the oligopeptides used in the hair treatment agents include leucine.

It is more preferred if the oligopeptides used in the hair treatment agents include isoleucine.

It is more preferred if the oligopeptides used in the hair treatment agents include arginine.

It is more preferred if the oligopeptides used in the hair treatment agents include valine.

Oligopeptides which are particularly preferred, or amino acid sequences included in the preferred oligopeptides, will be described below:

A particularly preferred oligopeptide additionally includes tyrosine, which is preferably bound via its acid function to the Glu-Glu-Glu sequence. Preferred hair treatment agents are therefore characterized in that the oligopeptide included therein comprises at least one Tyr-Glu-Glu-Glu amino acid sequence, where the amino group may be in free or protonated form and the carboxyl groups may be in free or deprotonated form.

A further particularly preferred oligopeptide additionally includes isoleucine, which is preferably bound via its amino function to the Glu-Glu-Glu sequence. Preferred hair treatment agents are therefore characterized in that the oligopeptide included therein comprises at least one Glu-Glu-Glu-Ile amino acid sequence, where the amino group may be in free or protonated form and the carboxyl groups may be in free or deprotonated form.

Oligopeptides which include both of the aforementioned amino acids (tyrosine and isoleucine) are preferred. In this case, hair treatment agents are particularly preferred in which the oligopeptide included therein comprises at least one Tyr-Glu-Glu-Glu-Ile amino acid sequence, where the amino group may be in free or protonated form and the carboxyl groups may be in free or deprotonated form.

More preferred oligopeptides additionally include arginine, which is preferably bound to isoleucine.

Accordingly, hair treatment agents are more preferred in which the oligopeptide included therein comprises at least one Tyr-Glu-Glu-Glu-Ile-Arg amino acid sequence, where the amino groups may be in free or protonated form and the carboxyl groups may be in free or deprotonated form.

Yet more preferred oligopeptides additionally include valine, which is preferably bound to the arginine. More preferred hair treatment agents are therefore characterized in that the oligopeptide included therein comprises at least one Tyr-Glu-Glu-Glu-Ile-Arg-Val amino acid sequence, where the amino groups may be in free or protonated form and the carboxyl groups may be in free or deprotonated form.

Yet more preferred oligopeptides additionally include leucine, which is preferably bound to the valine. More preferred hair treatment agents are characterized in that the oligopeptide included therein comprises at least one Tyr-Glu-Glu-Glu-Ile-Arg-Val-Leu amino acid sequence, where the amino groups may be in free or protonated form and the carboxyl groups may be in free or deprotonated form.

Particularly preferred oligopeptides additionally include leucine, which is preferably bound to the tyrosine. More preferred hair treatment agents are characterized in that the oligopeptide included therein comprises at least one Leu-Tyr-Glu-Glu-Glu-Ile-Arg-Val-Leu amino acid sequence, where the amino groups may be in free or protonated form and the carboxyl groups may be in free or deprotonated form.

Very particularly preferably, the agents include at least two oligopeptides that satisfy the above-mentioned criteria but differ from one another. Thus, for example, hair treatment agents are preferred that include at least two oligopeptides A and B that are different from one another and that both include the Glu-Glu-Glu amino acid sequence.

Such oligopeptides A and B that are different from one another correspond to each other in that they bear three consecutive Glu-amino acids in their amino acid sequence, but have differences in the amino acids bound before or after. Peptides that are different from one another but correspond in part and that can be significantly larger than in the three amino acids mentioned above are preferred.

Thus, more preferred hair treatment agents are characterized in that they include at least two oligopeptides A and B that are different from one another and that both include the Glu-Glu-Glu-Ile amino acid sequence.

Hair treatment agents that include at least two oligopeptides A and B that are different from one another and that both include the Tyr-Glu-Glu-Glu amino acid sequence are likewise preferred.

Hair treatment agents that are likewise more preferred include at least two oligopeptides A and B that are different from one another and that both include the Tyr-Glu-Glu-Glu-Ile amino acid sequence.

Very particularly preferred hair treatment agents are characterized in that they include at least two oligopeptides A and B that are different from one another and that both include the Glu-Glu-Glu-Ile-Arg amino acid sequence.

Hair treatment agents that are likewise very particularly preferred include at least two oligopeptides A and B that are different from one another and that both include the Tyr-Glu-Glu-Glu-Ile-Arg amino acid sequence.

There is preferably an even greater structural correspondence among the oligopeptides. Thus, hair treatment agents that include at least two oligopeptides A and B that are different from one another and that both include the Glu-Glu-Glu-Ile-Arg-Val amino acid sequence are more preferred embodiments of the present invention.

Hair treatment agents that include at least two oligopeptides A and B that are different from one another and that both include the Tyr-Glu-Glu-Glu-Ile-Arg-Val amino acid sequence are likewise preferred embodiments.

Even more preferred hair treatment agents include at least two oligopeptides A and B that are different from one another and that both include the Glu-Glu-Glu-Ile-Arg-Val-Leu amino acid sequence.

Hair treatment agents that are likewise even more preferred include at least two oligopeptides A and B that are different from one another and that both include the Tyr-Glu-Glu-Glu-Ile-Arg-Val-Leu amino acid sequence.

Particularly preferred hair treatment agents are characterized in that they include at least two oligopeptides A and B that are different from one another, the oligopeptide A comprising the Leu-Tyr-Glu-Glu-Glu-Ile-Arg-Val-Leu amino acid sequence, where the amino groups may be in free or protonated form and the carboxyl groups may be in free or deprotonated form, and the oligopeptide B comprising the Tyr-Glu-Glu-Glu-Ile-Arg-Val-Leu amino acid sequence, where the amino groups may be in free or protonated form and the carboxyl groups may be in free or deprotonated form.

Very particularly preferred agents of this last-mentioned embodiment include—based on the weight of the agent—0.00001 to 1 wt. % oligopeptide A and 0.00001 to 1 wt. % oligopeptide B.

More preferred agents of this last-mentioned embodiment include—based on the weight of the agent—0.00005 to 0.1 wt. % oligopeptide A and 0.00005 to 0.1 wt. % oligopeptide B.

Even more preferred agents of this last-mentioned embodiment include—based on the weight of the agent—0.0001 to 0.01 wt. % oligopeptide A and 0.0001 to 0.001 wt. % oligopeptide B.

The oligopeptides that are used within the context of the present invention and that satisfy the above-mentioned requirements can advantageously be obtained from keratinous materials. These oligopeptides are known by the INCI name: “Hydrolyzed keratin”.

The hair treatment agents include the oligopeptide that comprises at least one Glu-Glu-Glu amino acid sequence preferably in a quantity of 0.001 to 10.0 wt. %, more preferably 0.01 to 5.0 wt. % and particularly preferably 0.05 to 1.0 wt. %, based in each case on the weight of the ready-to-apply hair treatment agent.

The hair treatment agents include—based on their weight—0.01 to 5.0 wt. % magnesium citrate as a second essential ingredient.

Magnesium citrate can be present in various forms. These forms can include hydrous trimagnesium dicitrate, water-free trimagnesium dicitrate, hydrous magnesium hydrogen citrate or water-free magnesium hydrogen citrate. Particular preferably, the magnesium citrate used includes is water-free trimagnesium dicitrate (Mg₃(C₆HSO₇)₂, CAS-No.: 3344-18-1), trimagnesium dicitrate nonahydrate (Mg₃(C₆HSO₇)₂×9H₂O, CAS-No.: 153531-96-5) or magnesium hydrogen citrate (C₆H₆O₇Mg, CAS-No.: 7779-25-1).

The hair treatment agents can be prepared using each of these forms individually or a combination of two or more of these magnesium citrate forms. Accordingly, it is preferred for a hair treatment agent to be obtained using water-free trimagnesium dicitrate and/or trimagnesium dicitrate nonahydrate and/or water-free magnesium hydrogen citrate and/or hydrous magnesium hydrogen citrate. It is particularly preferred for the hair treatment agent to be obtained using water-free trimagnesium dicitrate.

The hair treatment agents include magnesium citrate preferably in a quantity of 0.5 to 4.0 wt. % and particularly preferably 1.0 to 2.0 wt. %, based in each case on the weight of the ready-to-apply hair treatment agent. It is particularly preferred for 1.0 to 2.0 wt. % water-free trimagnesium dicitrate, based on the weight of the ready-to-apply hair treatment agent, to be used when preparing the hair treatment agent.

The hair treatment agent has a pH in the range of 3.5 to 5.5. The pH is preferably from 3.75 to 5.25, more preferably from 4.0 to 5.0 and in particular from 4.2 to 4.7. After several treatment processes, the color change in artificially obtained hair colors is particularly small, the care performance at the same time being good, if the hair treatment agent has a pH in the range of 4.3 to 4.7.

If necessary, the pH is adjusted using suitable pH-adjusting agents such as acids or bases, after mixing the further ingredients of the hair treatment agent.

It has been found that using the combination of selected oligopeptides and magnesium citrate, preferably water-free magnesium hydrogen citrate, provides the hair treatment agents with outstanding properties. Thus, the color change in artificially obtained hair colors is extremely low after several treatment processes. In addition, the hair treatment agents make the hairs treated therewith more resilient, which is reflected in higher tensile strengths of the keratin fibers and in a lessening of the reduction in elasticity, for example when damaged by atmospheric influences. In addition, the moisture balance of the keratin fibers is stabilized, with the result that the combability is improved and the aging process is slowed.

The hair treatment agents can in principle be used on hair that has been colored using permanent, semi-permanent or temporary hair dyes. Temporary hair dyes are, however, intended to be washed out and/or to fade over time, and therefore the hair treatment agent is particularly suitable for use on hair that has been colored using permanent or oxidative hair dyes.

The hair treatment agents can include further active ingredients and auxiliary agents. These are described in the following.

Preferably, the hair treatment agents also include at least one surfactant, the surface-active substances being referred to as surfactants or emulsifiers, depending on the field of use, and being selected from anionic, cationic, amphoteric/zwitterionic and non-ionic surfactants and emulsifiers.

Preferred hair treatment agents are characterized in that they include—based on their weight—0.5 to 70 wt. %, preferably 1 to 60 wt. % and in particular 5 to 25 wt. % anionic and/or non-ionic and/or cationic and/or amphoteric/zwitterionic surfactant(s).

All anionic surface-active substances suitable for use on the human body are suitable as anionic surfactants for the hair treatment agents. They are characterized by a water-solubilizing anionic group, such as a carboxylate, sulfate, sulfonate, or phosphate group, and a lipophilic alkyl group having approximately 8 to 30 C atoms. The molecule may also include glycol groups or polyglycol ether groups, ester, ether and amide groups as well as hydroxyl groups.

The term “amphoteric/zwitterionic surfactants” refers to those surface-active compounds that bear at least one quaternary ammonium group and at least one —COO⁽⁻⁾ group or —SO3₃⁽⁻⁾ group in the molecule. Especially suitable zwitterionic surfactants and emulsifiers are the betaines, such as the N-alkyl-N,N-dimethylammonium glycinates, for example coco alkyl dimethylammonium glycinate, N-acyl-aminopropyl-N,N-dimethylammonium glycinates, for example coco acyl aminopropyl dimethylammonium glycinate, and 2-alkyl-3-carboxymethyl-3-hydroxyethyl imidazolines having 8 to 18 C atoms in the alkyl or acyl group in each case, and coco acyl aminoethyl hydroxyethyl carboxymethyl glycinate. A preferred zwitterionic surfactant is the fatty acid amide derivative known by the INCI name cocamidopropyl betaine.

“Amphoteric/zwitterionic surfactants” also refers to those surface-active compounds that include at least one free amino group and at least one —COOH— or —SO₃H— group in addition to a C₈-C₂₄ alkyl or acyl group, and are capable of forming zwitterions. Examples of suitable amphoteric/zwitterionic surfactants are N-alkylglycines, N-alkylaminopropanoic acids, N-alkylaminobutyric acids, N-alkyliminodipropanoic acids, N-hydroxyethyl-N-alkylamidopropylglycines, N-alkyltaurines, N-alkylsarcosines, 2-alkylaminopropanoic acids and alkylaminoacetic acids, each having approximately 8 to 24 C atoms in the alkyl group. Particularly preferred amphoteric/zwitterionic surfactants are N-cocoalkylaminopropionate, cocoacylaminoethylamino propionate, and C₁₂-C₁₈ acyl sarcosine.

Non-ionic surfactants and emulsifiers include for example a polyol group, a polyalkylene glycol ether group or a combination of a polyol group and a polyglycol ether group as the hydrophilic group.

Very particularly preferable are agents that preferably include, in addition to fatty alcohol(s) and/or fatty alcohol ethoxylate(s), C₁₂₋₂₂ fatty alcohol(s) and/or C₁₂₋₂₂ fatty alcohol ethoxylate(s) having 10 to 30 EO units, particularly preferably C₁₆₋₁₈ fatty alcohol(s) and/or C₁₆₋₁₈ fatty alcohol ethoxylate(s) having 12 to 20 EO units, preferably in quantities of 5 to 20 wt. %, preferably 7.5 to 17.5 wt. % and in particular 10 to 15 wt. %, based in each case on the weight of the hair treatment agent.

In summary, hair treatment agents are preferred that include—based on their weight—0.1 to 20 wt. %, preferably 0.25 to 17.5 wt. % and in particular 5 to 15 wt. % anionic surfactant(s), particularly preferably fatty alcohol ether sulfates of the formula

H₃C—(CH₂)ₙ—(OCH₂CH₂)ₖ—OSO₃⁻M⁺,

in which n represents values of 5 to 21, preferably 7 to 19, particularly preferably 9 to 17 and in particular 11 to 13, and k represents values of 1, 2, 3, 4, 5, 6, 7, 8, 9 or 10, preferably 1, 2 or 3 and in particular 2, and M represents a cation from the group Na⁺, K⁺NH₄⁺, ½ Mg²⁺, ½Zn²⁺, preferably Na⁺.

As a further optional component, the hair treatment agents can include 0.01 to 10 wt. % of at least one polymer from the group of the cationic and/or amphoteric polymers.

Cationic or amphoteric polymers are to be understood as polymers which comprise a group, in the main chain and/or side chain, that can be “temporarily” or “permanently” cationic. The term “permanently cationic” refers to those polymers that comprise a cationic group irrespective of the pH of the agent. These are generally polymers that include a quaternary nitrogen atom, for example in the form of an ammonium group. Preferred cationic groups are quaternary ammonium groups. In particular those polymers in which the quaternary ammonium group is bound via a C₁₋₄ hydrocarbon group to a polymer main chain composed of acrylic acid, methacrylic acid or derivatives thereof have proven particularly suitable.

Preferred cationic polymers are selected from
poly(methacryloyloxyethyl trimethylammonium chloride) (INCI: Polyquaternium-37) and/or;
quaternized cellulose derivatives (INCI: Polyquaternium 10) and/or
cationic alkyl polyglycosides and/or
cationized honey and/or
cationic guar derivatives and/or
polymeric dimethyldiallylammonium salts and the copolymers thereof with esters and amides of acrylic acid and methacrylic acid, and/or
copolymers of vinylpyrrolidone with quaternized derivatives of dialkyl aminoalkyl acrylate and dialkyl aminoalkyl methacrylate, and/or
vinyl pyrrolidone-vinyl imidazolium methochloride copolymers and/or
quaternized polyvinyl alcohol and/or
Polyquaternium-2 and/or
Polyquaternium-6 and/or
Polyquaternium-7 and/or
Polyquaternium-17 and/or
Polyquaternium-18 and/or
Polyquaternium-24 and/or
Polyquaternium-27 and/or
Polyquaternium-101.

In addition to cationic polymerisates, or in place thereof, the hair treatment agents can also include amphoteric polymers. These have, in addition to the cationically charged group(s), at least one negatively charged group in the molecule and are also referred to as zwitterionic polymers.

The polymer or polymers is/are preferably used within narrow quantity ranges. Thus, agents are preferred which include—based on their weight—0.05 to 7.5 wt. %, preferably 0.1 to 5 wt. %, particularly preferably 0.2 to 3.5 wt. % and in particular 0.25 to 2.5 wt. % amphoteric polymer(s).

Irrespective of whether or not the agents include amphoteric polymers, more preferred agents are characterized in that they include—based on their weight—0.05 to 7.5 wt. %, preferably 0.1 to 5 wt. %, particularly preferably 0.2 to 3.5 wt. % and in particular 0.25 to 2.5 wt. % cationic polymer(s).

The silicones are a further group of possible ingredients. Hair treatment agents preferably include silicone(s) in quantities of 0.1 to 10 wt. %, preferably 0.25 to 7 wt. % and in particular 0.5 to 5 wt. %, based in each case on the agent as a whole.

Further suitable ingredients include non-ionic polymers, fatty substances, waxes, proteolipids, amino acids, vitamins, provitamins, vitamin precursors, betaines, bioquinones, purine (derivatives), taurine (derivatives), L-carnitine (salts), panthenol, pantothenic acid, 2-furanone, 2-furanone derivatives, ectoine, allantoin, plant extracts, ester oils, UV filters, structuring agents, thickening agents, electrolytes, pH-adjusting agents, swelling agents, dyes, anti-dandruff active ingredients, complexing agents, opacifying agents, pearlescing agents, pigments, stabilizers, propellants, antioxidants, perfume oils and/or preservatives.

It is particularly preferred for the further ingredient of the hair treatment agent to be citric acid.

The present invention also relates to a method for reducing and/or preventing the bleeding and/or lightening of artificially obtained hair colors, and/or for improving the color intensity and/or the color fidelity, and for improving at least one of the properties of
tensile strength of keratin fibers, in particular human hair;
stabilizing the moisture balance of keratin fibers, in particular human hair;
combability of keratin fibers, in particular human hair;
increasing the contact angle between water droplets and the surface of keratin fibers, in particular human hair;
lessening the reduction in elasticity of keratin fibers, in particular human hair, when damaged by atmospheric influences,
wherein a hair treatment agent that has a pH of between 3.5 and, 5.5 and can be obtained by mixing
   a) to 20 wt. %—based on the total weight of the hair treatment agent—of at least one oligopeptide that comprises at least one Glu-Glu-Glu amino acid sequence,

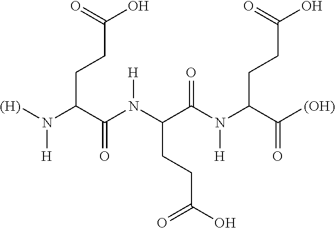

where the amino group may be in free or protonated form and the carboxyl groups may be in free or deprotonated form, and
   b) to 5.0 wt. %—based on the total weight of the hair treatment agent—of magnesium citrate in a hydrous carrier,
      is applied to the keratin fibers, left there for between 5 seconds and 10 minutes, and then rinsed out, or
      is applied to the keratin fibers and remains there.

That which has been stated regarding the hair treatment agents applies, mutatis mutandis, to additional preferred embodiments of the method.

The following examples are intended to explain the subject matter of the present invention, but without restricting said subject matter.

### Examples

Unless otherwise specified, all the stated quantities are parts by weight of the active substance. The following formulations were provided using known preparation methods.

Hair Conditioners 1:

| | wt. % | | | |
|---|---|---|---|---|
| | 1A | 1B | 1C | 1D |
| Cetearyl alcohol | 5.0 | 5.0 | 5.0 | 5.0 |
| Quaternium-87 | 0.75 | 0.75 | 0.75 | 0.75 |
| Glycol distearate | 1.0 | 1.0 | 1.0 | 1.0 |
| Hydrolyzed keratin* | 0.1 | 0.1 | 0.1 | 0.1 |
| Shea butter (INCI: Butyrospermum | 1.5 | 1.5 | 1.5 | 1.5 |
| Parkii (Shea) Butter) | | | | |
| Water-free trimagnesium dicitrate | 1.0 | 1.5 | 1.0 | 1.0 |
| Lactic acid | 0.1 | 0.1 | 0.1 | 0.1 |
| Behenoyl PG-trimoniumchloride | 1.5 | 1.5 | 1.5 | 1.5 |
| Behentrimonium chloride | 0.5 | 0.5 | 0.5 | 0.5 |
| Polyquaternium-37 | 0.4 | 0.4 | 0.4 | 0.4 |
| Amodimethicone/ | — | 0.2 | — | — |
| morpholinomethyl | | | | |
| silsesquioxane copolymer | | | | |
| Polyquaternium-101 | — | — | 0.2 | — |
| Polyquaternium-10 | — | — | — | 0.2 |
| Glycerol | 0.1 | 0.1 | 0.1 | 0.1 |
| Dimethicone | 1.0 | 0.5 | 0.5 | 1.0 |
| Water, preservative, accompanying | to 100 | to 100 | to 100 | to 100 |
| substances and optionally perfume | | | | |
| oils | | | | |

Care Spray 2:

| | wt. % |
|---|---|
| Stearamidopropyl dimethylamine | 0.3 |
| Glycerol | 2.0 |
| Polyquaternium-10 | 0.2 |
| Hydrolyzed keratin* | 0.1 |
| Panthenol | 0.2 |
| Water-free trimagnesium dicitrate | 1.0 |
| Ethanol | 15 |
| Nicotinamide | 0.1 |
| Dicaprylyl carbonate | 0.1 |
| Polyquaternium-72 | 0.2 |
| Cetrimonium chloride | 0.2 |
| Water, preservative, accompanying substances and | to 100 |
| optionally perfume oils | |

Hair Shampoos 3:

| | wt. % | | | | |
|---|---|---|---|---|---|
| | 3A | 3B | 3C | 3D | 3E |
| Sodium laureth sulfate | 9.0 | 9.0 | 9.0 | 9.0 | 9.0 |
| Ammonium lauryl sulfate | 5.0 | 5.0 | 5.0 | 5.0 | 5.0 |
| Disodium | 2.0 | 2.0 | 2.0 | 2.0 | 2.0 |
| cocoamphodiacetate | | | | | |
| Cocoamidopropyl betaine | 6.0 | 6.0 | 6.0 | 6.0 | 6.0 |
| Water-free trimagnesium | 1.5 | 1.5 | 1.5 | 1.5 | 1.5 |
| dicitrate | | | | | |
| Panthenol | 0.2 | 0.2 | 0.2 | 0.2 | 0.2 |
| Hydrolyzed keratin* | 0.3 | 0.3 | 0.3 | 0.3 | 0.3 |
| Calcium lactate | 0.4 | 0.4 | 0.4 | 0.4 | — |
| Guar | 0.4 | 0.6 | 0.4 | 0.6 | 0.6 |
| hydroxypropyltrimonium | | | | | |
| chloride | | | | | |
| PEG 12 dimethicone | — | 0.3 | — | — | — |
| Polyquaternium-6 | — | — | 0.5 | — | — |
| Polyquaternium-7 | — | — | — | 0.4 | — |
| Polyquaternium-67 | — | — | — | — | 0.4 |
| Sodium chloride | 1.3 | — | 1.2 | 1.3 | 1.3 |
| Water, preservative and | to 100 | to 100 | to 100 | to 100 | to 100 |
| optionally perfume oils | | | | | |

*oligopeptide that comprises at least one Glu-Glu-Glu amino acid sequence

The pH of all the conditioners, care sprays and hair shampoos was between 4.3 and 4.5.

The agents were prepared using conventional methods, by mixing the ingredients in a hydrous carrier and subsequently adjusting the pH.

The following commercial products were used:

| | | Supplier/ |
|---|---|---|
| Commercial product | INCI | manufacturer |
| Lanette O | Cetearyl alcohol | BASF SE |
| Varisoft W575 PG | Quaternium-87 | Evonik |
| Cutina GMS-V or | Glycol distearate | BASF SE |
| Cutina AGS | | |
| Polymer JR400 | Polyquaternium-10 | Dow |
| Dehyquart A CA | Cetrimonium chloride | BASF SE |
| Synthalen ® CR | Polyquaternium-37 | 3V Sigma |
| Quartamin BTC 131 | Behenoyl PG-trimoniumchloride | Kao Chemicals |
| Genamin KDMP | Behentrimonium chloride | Clariant |
| Tego Amid S 18 | Stearamidopropyl dimethylamine | Evonik |
| Ucare ® Polymer | Polyquaternium-10 | Dow |
| JR 400 | | |
| Cetiol CC | Dicaprylyl carbonate | BASF SE |
| Texapon N70 NA | Sodium laureth sulfate | BASF SE |
| Empicol AL 70 | Ammonium lauryl sulfate | Huntsman |
| Rewoteric AM 2 | Disodium cocoamphodiacetate | Evonik |
| Tego Betain F50 | Cocoamidopropyl betaine | Evonik |
| N-Hance 3196 | Guar hydroxypropyltrimonium | Ashland |
| | chloride | |
| ProSina | Hydrolyzed keratin | Croda |
| Belsil ADM 8301 E | Amodimethicone/ | Wacker |
| | morpholinomethyl | |
| | silsesquioxane copolymer | |
| Deposilk Q1 | Polyquaternium-101 | Air Products |
| Xiameter OFX | PEG 12 dimethicone | Dow |
| 0193 Fluid | | |
| Merquat 100 | Polyquaternium-6 | Lubrizol |
| Merquat 550 | Polyquaternium-7 | Lubrizol |
| SoftCAT Polymer | Polyquaternium-67 | Dow |
| SX 400 X | | |
| Mirustyle CP LQ | Polyquaternium-72 | Croda |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent having a pH of between 3.5 and 5.5 and including, in a hydrous carrier:
a) 0.01 to 20 wt. %—based on the total weight of the hair treatment agent—at least one oligopeptide that comprises at least one Glu-Glu-Glu amino acid sequence, 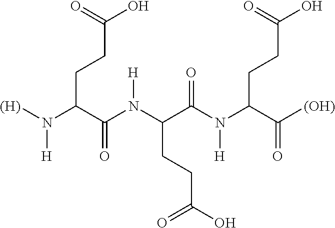
wherein the amino group is in free or protonated form and the carboxyl groups are in free or deprotonated form, and
b) 0.01 to 5.0 wt. %—based on the total weight of the hair treatment agent—of magnesium citrate.

2. The hair treatment agent according to claim 1, wherein the oligopeptide includes 5 to 15 amino acids and has a molecular weight of 650 to 3000 Da.

3. The hair treatment agent according to claim 1, wherein the oligopeptide includes Tyr-Glu-Glu-Glu-Ile, 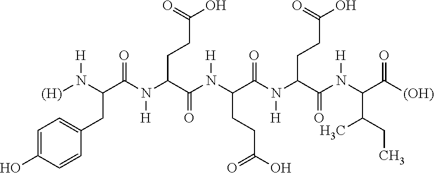
wherein the amino group is in free or protonated form and the carboxyl groups are in free or deprotonated form.

4. The hair treatment agent according to claim 1, wherein the oligopeptide includes at least one Tyr-Glu-Glu-Glu-Ile-Arg-Val-Leu amino acid sequence, 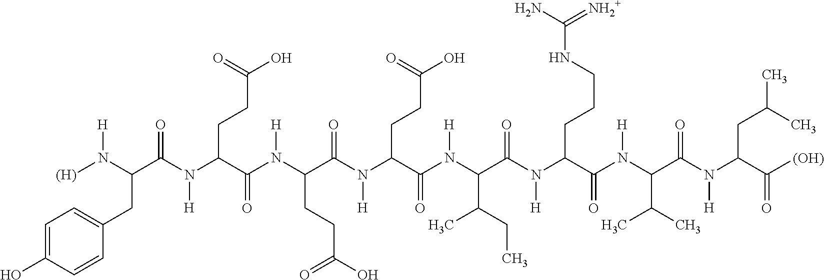
wherein the amino groups are in free or protonated form and the carboxyl groups are in free or deprotonated form.

5. The hair treatment agent according to claim 1, wherein the magnesium citrate is selected from the group consisting of water-free trimagnesium dicitrate, hydrous trimagnesium dicitrate, water-free magnesium hydrogen citrate, hydrous magnesium hydrogen citrate and mixtures thereof.

6. The hair treatment agent according to claim 5, wherein the magnesium citrate is water-free trimagnesium dicitrate.

7. The hair treatment agent according to claim 1, further including—based on its weight—0.5 to 70 wt. % anionic and/or non-ionic and/or cationic and/or amphoteric surfactant(s).

8. The hair treatment agent according to claim 1, further including—based on its weight—0.05 to 7.5 wt. % at least one cationic polymer selected from the group consisting of
polyquaternium-37;
polyquaternium 10;
cationic alkyl polyglycosides;
cationized honey;
cationic guar derivatives;
polymeric dimethyldiallylammonium salts and the copolymers thereof with esters and amides of acrylic acid and methacrylic acid;
copolymers of vinylpyrrolidone with quaternized derivatives of dialkyl aminoalkyl acrylate and dialkyl aminoalkyl methacrylate;
vinylpyrrolidone-vinyl imidazolium methochloride copolymers;
quaternized polyvinyl alcohol;
polyquaternium-2;
polyquaternium-6;
polyquaternium-7;
polyquaternium-17;
polyquaternium-18;
polyquaternium-24;
polyquaternium-27; and
polyquaternium-101.

9. The hair treatment according to claim 1, wherein the pH of the hair treatment agent is between 4.3 and 4.7.

10. A method for reducing and/or preventing the bleeding and/or fading of artificially obtained hair colors, and/or for improving the color intensity and/or the color fidelity, and for improving at least one of the properties of
tensile strength of keratin fibers, in particular human hair;
stabilizing the moisture balance of keratin fibers, in particular human hair;
combability of keratin fibers, in particular human hair;
increasing the contact angle between water droplets and the surface of keratin fibers, in particular human hair;
lessening the reduction in elasticity of keratin fibers, in particular human hair, when damaged by atmospheric influences,
the method including:
applying to the keratin fibers a hair treatment agent that has a pH of between 3.5 and 5.5 and is obtained by mixing in a hydrous carrier
a) 0.01 to 20 wt. %—based on the total weight of the hair treatment agent—of at least one oligopeptide that comprises at least one Glu-Glu-Glu amino acid sequence, 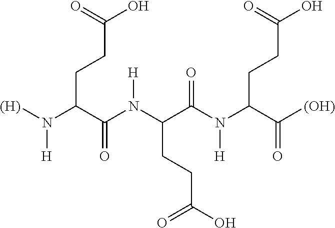
wherein the amino group is in free or protonated form and the carboxyl groups are in free or deprotonated form, and
b) to 5.0 wt. %—based on the total weight of the hair treatment agent—magnesium citrate.
